(19) United States
(12) Patent Application Publication
(10) Pub. No.: US 2009/0204089 A1
(43) Pub. Date: Aug. 13, 2009

(54) Postpartum Pad

(76) Inventors: Douglas F. Caldwell, Nevada City, CA (US); Pamela C. Caldwell, Nevada City, CA (US)

Correspondence Address: NEUSTEL LAW OFFICES, LTD., 2534 SOUTH UNIVERSITY DRIVE, SUITE 4, FARGO, ND, 58103, US

(21) Appl. No.: 12/030,261

(22) Filed: Feb. 13, 2008

(51) Int. Cl. A61K 36/18; A61F 13/40; A61P 43/00; A61F 13/551

(52) U.S. Cl. 604/385.02; 604/385.06; 424/725

(57) ABSTRACT

A postpartum pad for efficiently soothing and healing a woman's perineal area after birth. The postpartum pad generally includes a pad including at least one reservoir portion, wherein the at least one reservoir portion extends within an upper surface of the pad and a liquid extract used for healing or soothing of wounds positioned within the reservoir portion. At least a portion of the pad is comprised of an absorbent material to absorb up any excess liquid extract or fluid from the perineal and/or vaginal area of the woman.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to postpartum devices and more specifically it relates to a postpartum pad for efficiently soothing and healing a woman's perineal area after birth.

2. Description of the Related Art

Any discussion of the related art throughout the specification should in no way be considered as an admission that such related art is widely known or forms part of common general knowledge in the field.

There are millions of pregnant women every year in the United States and throughout the world. Generally, when a woman goes the process of giving birth to a child, the woman may experience substantial pain (i.e. leading from tears, etc.) in the woman's perineal area (i.e. perineum and including vaginal area), wherein this pain generally continues for a period of time after giving birth to the child.

Various devices have been tried to overcome the pains and discomforts associated with the women's perineal area after childbirth. One such device, sometimes provided by hospitals, is a latex glove, rubber-banded closed and frozen, The woman is to apply the latex glove to their perineal area to help soothe the pain. However, the latex glove is generally comprised of an awkward shape and must be held in place manually in order to maintain contact with the injured area. The latex glove also generally does nothing for healing of the perineal area. In addition, many women are also sensitive to latex which may add to further discomforts.

Another product that is available to try and soothe the women's perineal area after childbirth is chemically-activated cold packs. However, the cold packs also do nothing for healing the perineal area and further include chemical components, wherein most mothers may most likely want to avoid chemical components after childbirth because of the danger of infection or other damaging effects.

There are also bulk herb combinations available for a woman to use on their perineal area after childbirth. However, these herb combinations are generally time-consuming and confusing in that the woman must prepare the pads with the herb combinations themselves. These herb combinations are especially ineffective if the woman gives birth much earlier than expected, wherein the pad and herb combinations may not have even been prepared yet. Because of the inherent problems with the related art, there is a need for a new and improved postpartum pad for efficiently soothing and healing a woman's perineal area after birth.

### BRIEF SUMMARY OF THE INVENTION

The general purpose of the present invention is to provide a postpartum pad that has many of the advantages of the postpartum devices mentioned heretofore. The invention generally relates to a postpartum device which includes a pad including at least one reservoir portion, wherein the at least one reservoir portion extends within an upper surface of the pad and a liquid extract used for healing or soothing of wounds positioned within the reservoir portion. At least a portion of the pad is comprised of an absorbent material to absorb up any excess liquid extract or fluid from the perineal and/or vaginal area of the woman.

There has thus been outlined, rather broadly, some of the features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction or to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

An object is to provide a postpartum pad for efficiently soothing and healing a woman's perineal area after birth.

Another object is to provide a postpartum pad that is commercially available in a pre-made form.

An additional object is to provide a postpartum pad that will help reduce pain, swelling, and will assist in the healing process of the woman's perineal area.

A further object is to provide a postpartum pad that is comprised of an absorbent material.

Another object is to provide a postpartum pad that will be prepackaged and thus require no preparation before utilizing.

Another object is to provide a postpartum pad that can be easily frozen to provide pain relief and to minimize swelling of the perineal and/or vaginal area.

Another object is to provide a postpartum pad that will be individually wrapped to ensure purity and freshness, and to avoid pads from sticking together when frozen.

Another object is to provide a postpartum pad that will be pre-formed to more comfortably fit the woman's perineal area.

Another object is to provide a postpartum pad that will be 100% organic and bacteria free.

Another object is to provide a postpartum pad that is infused with healing liquids (e.g. Witch Hazel, etc.).

Another object is to provide a postpartum pad that is disposable.

Other objects and advantages of the present invention will become obvious to the reader and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is a top view of the present invention.

FIG. 3 is a side view of the present invention.

FIG. 4 is a side cross-sectional view of the present invention.

FIG. 5 is an upper perspective view of the present invention within a packaging.

FIG. 6 is an upper perspective view of the present invention within a packaging and stored within a container.

### DETAILED DESCRIPTION OF THE INVENTION

### A. Overview

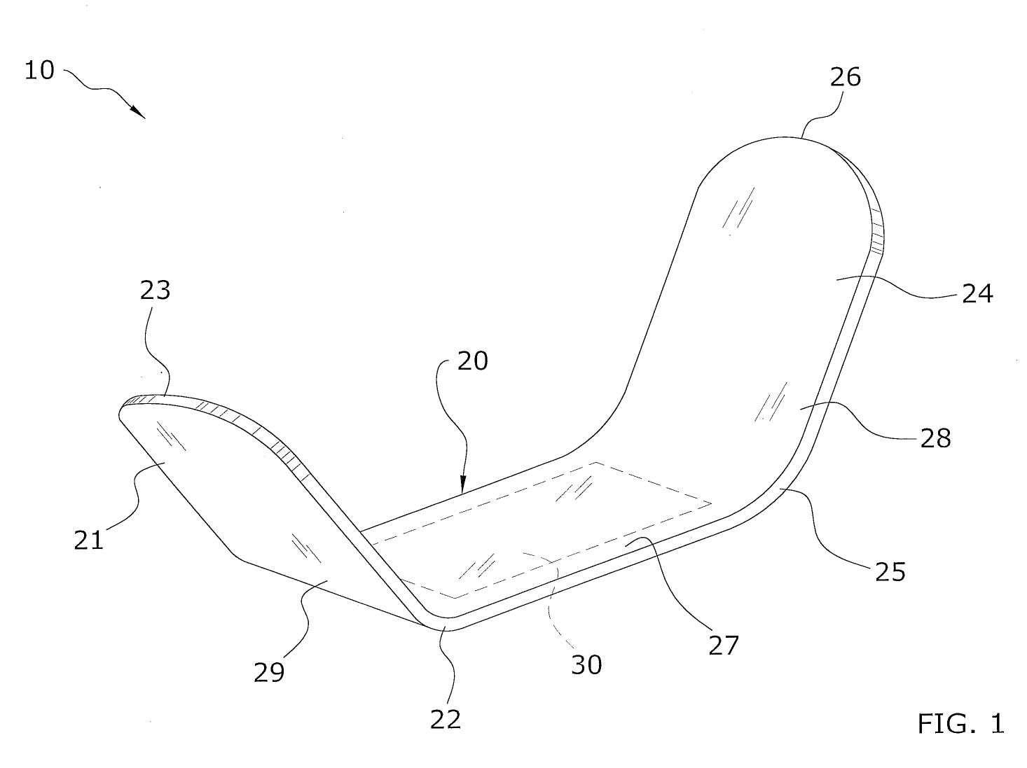

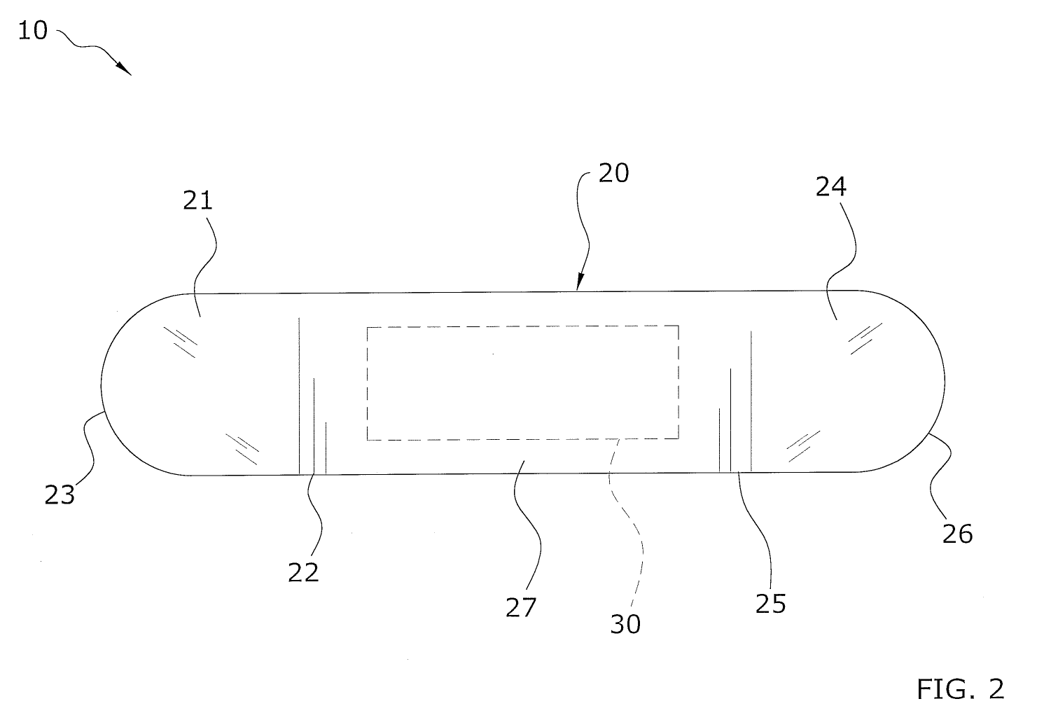

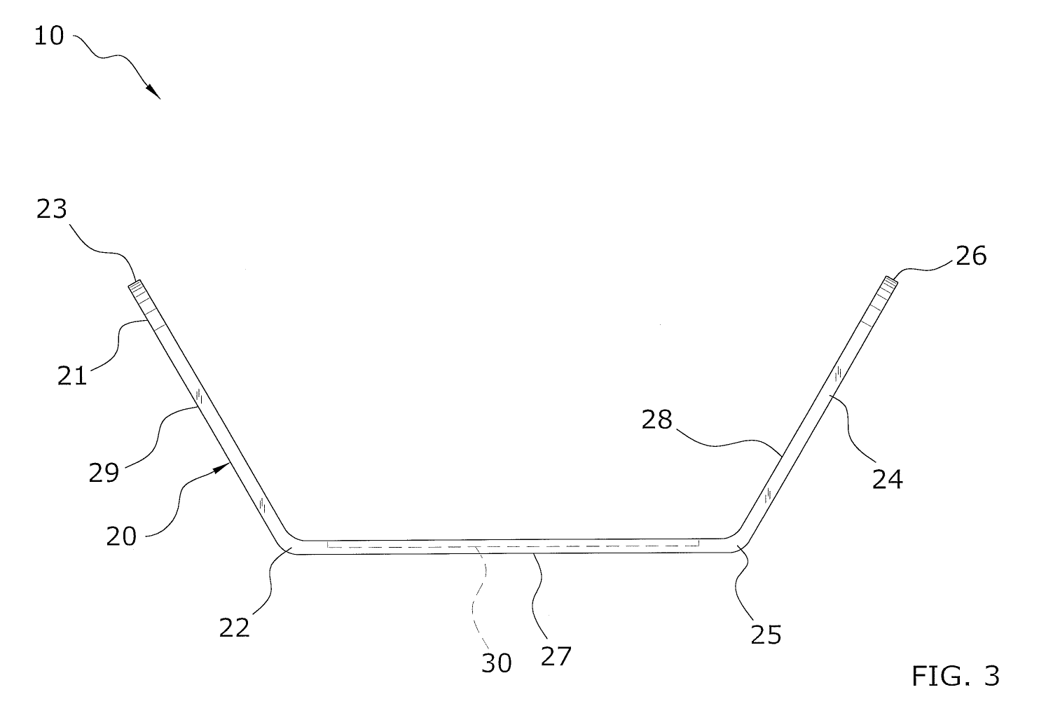

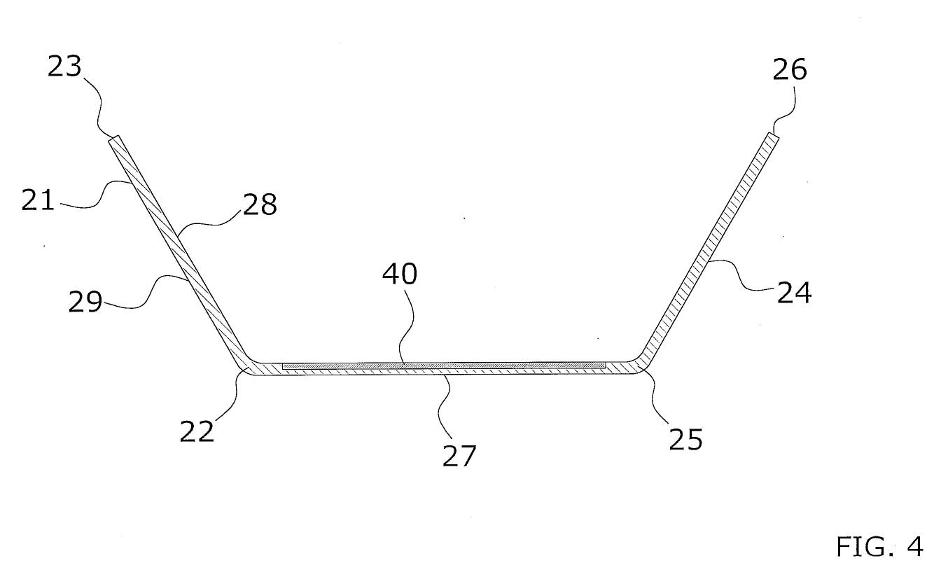

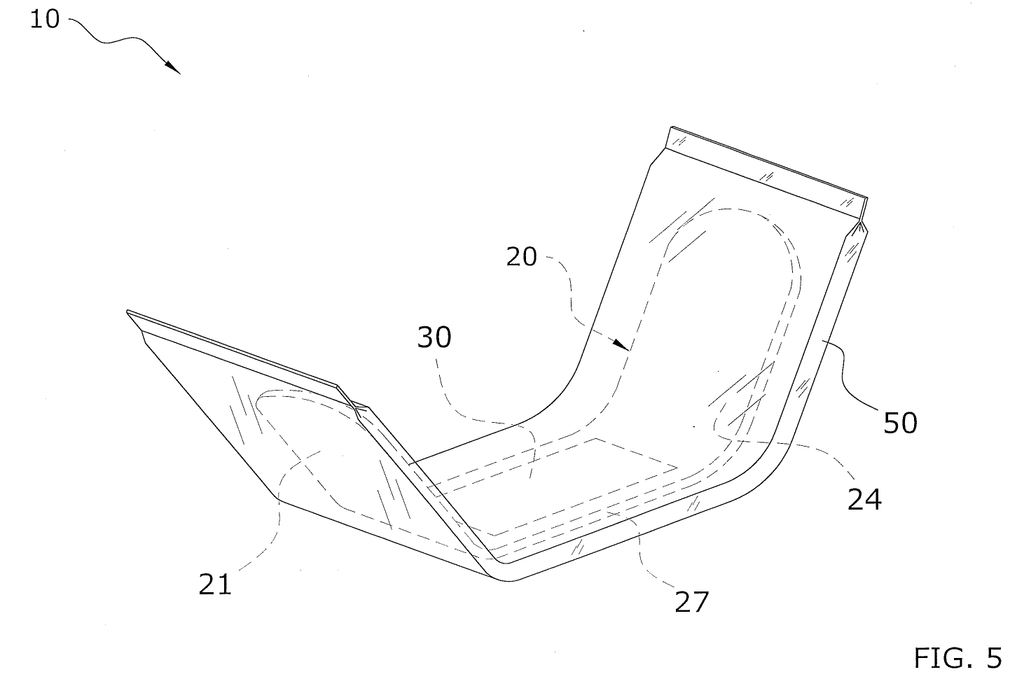

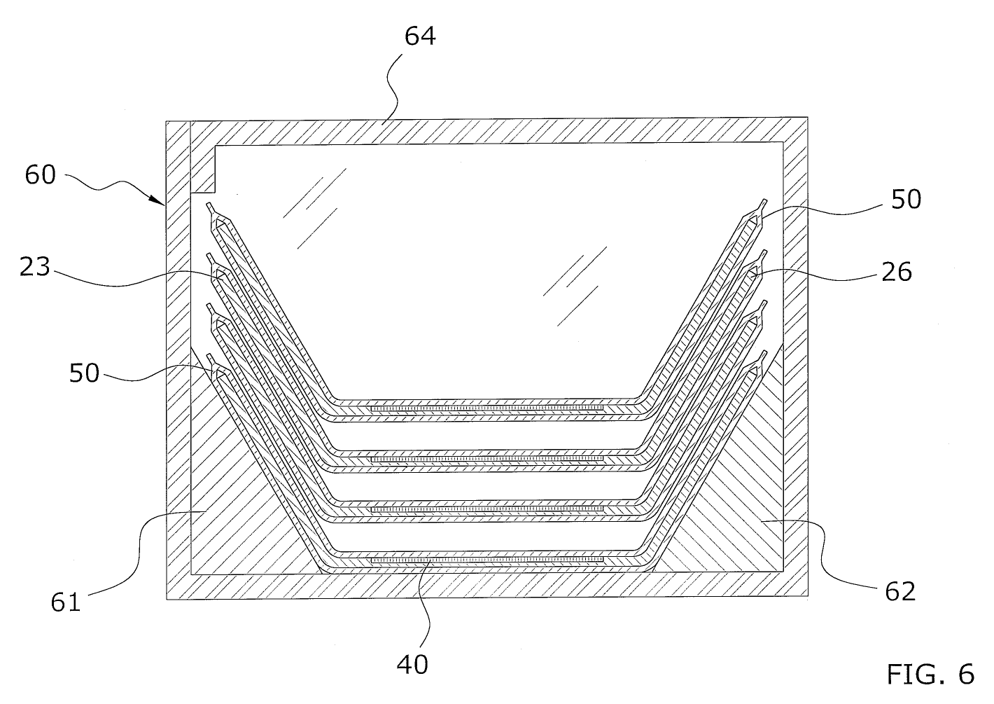

Turning now descriptively to the drawings, in which similar reference characters denote similar elements throughout the several views, FIGS. 1 through 6 illustrate a postpartum pad 10, which comprises a pad 20 including at least one reservoir portion 30, wherein the at least one reservoir portion 30 extends within an upper surface 28 of the pad 20 and a liquid extract 40 used for healing or soothing of wounds positioned within the reservoir portion 3. At least a portion of the pad 20 is comprised of an absorbent material to absorb up any excess liquid extract or fluid from the perineal and/or vaginal area of the woman.

### B. Pad

The pad 20 is comprised of a soft configuration that may be comfortably applied to a woman's perineal area. The material that the pad 20 is comprised of is preferably comprised of an organic material (e.g. 100% organic cotton, etc.) so to prevent any infections or reactions that the woman may have to the pad 20. The pad 20 is also preferably comprised of an absorbent material so as to soak up any fluids commonly associated with postpartum blood being secreted from the woman. The pad 20 is also comprised of a material that allows for the pad 20 to be frozen and thawed without disrupting the configuration of the pad 20. It is appreciated that freezing the pad 20 helps to reduce swelling, inflammation and pain of the perineal area of the woman.

The pad 20 is preferably comprised of a preformed shape so as to comfortably be worn be the woman without having to bend or flex the pad 20. Having the pad 20 preformed also preferably assists in maintaining the pad 20 in a proper position with respect to the woman. In the preferred embodiment of the present invention, the pad 20 is preferably comprised of a preformed U-shaped configuration to naturally fit the woman's body; however it is appreciated that the pad 20 may be comprised of various shapes according to desired application of the pad 20.

The pad 20 includes a first outer portion 21, a second outer portion 24 and a center portion 27 extending between the first outer portion 21 and the second outer portion 24. The first outer portion 21 and the second outer portion 24 help to secure the liquid extract 40 and the any other fluids upon the pad 20 by forming a barrier around the center portion 27 and the perineal area of the woman. The first outer portion 21 and the second outer portion also help to secure the pad 20 in place about the perineal area of the woman to prevent the pad 20 from moving around while in use. The first outer portion 21 and the second outer portion 24 further preferably perform a majority of the absorbing while utilizing the present invention, wherein the first outer portion 21 and the second outer portion 24 absorb any excess liquid extract 40 and fluids (e.g. bleeding, etc.) from the woman's perineal area.

The first outer portion 21, the center portion 27 and the second outer portion 24 are preferably comprised of an integral structure; however it is appreciated in various embodiments the first outer portion 21, the center portion 27 and the second outer portion 24 may be comprised of separate structures. The first outer portion 21, the center portion 27 and the second outer portion 24 also preferably include a uniformly smooth outer surface and inner surface so as to fit comfortably against the individual utilizing the present invention. A first outer end 23 of the first outer portion 21 and a second outer end 26 of the second outer portion 24 are also preferably rounded.

A first preformed bend 22 is preferably between the center portion 27 and the first outer portion 21. The first preformed bend 22 is preferably flexible so as to allow the first outer portion 21 to pivot about the center portion 27 if desired. The first preformed bend 22 also extends the first outer portion 21 at an upward angle with respect to the center portion 27. A second preformed bend 25 is preferably between the center portion 27 and the second outer portion 24. The second preformed bend 25 is preferably flexible so as to allow the second outer portion 24 to pivot about the center portion 27 if desired. The second preformed bend 25 also extends the second outer portion 24 at an upward angle with respect to the center portion 27. The first outer portion 21 and the second outer portion 24 further preferably mirror each other about the longitudinal center of the center portion 27.

The center portion 27 is preferably comprised of an elongated configuration and proper width to properly extend over the entire perineal area of the woman. The center portion 27 includes a reservoir portion 30 preferably centrally positioned upon the center portion 27. The reservoir portion 30 is preferably comprised of a permeable and absorbent configuration to retain the liquid extract 40.

The reservoir also preferably extends within a majority of the surface area of the center portion 27 to also cover the entire perineal area of the woman. The reservoir portion 30 preferably extends at least partially within the center portion 27 from the upper surface 28 of the pad 20. It is appreciated that the reservoir portion 30 need not extend all the way though the pad 20 to the lower surface 29, wherein the lower surface 29 is not in contact with the perineal area of the woman. It is appreciated that the pad 20 may include a plurality of reservoir portions 30 in alternate embodiments of the present invention. The reservoir portion 30 is also preferably concentric with the pad 20.

### C. Liquid Extract

The pad 20 includes a liquid extract 40 integrated with the pad 20. The liquid extract 40 may be comprised of various compositions all which provide comfort and healing to the woman and the woman's perineal area during use of the present invention. The liquid extract 40 is preferably positioned within the reservoir portion 30 of the pad 20. The liquid extract 40 is preferably frozen in the reservoir portion 30 of the pad 20 so as to be ready for use when desired.

In the preferred embodiment, the liquid extract 40 is preferably comprised of witch hazel extract. It is appreciated that the pad 20 may be manufactured with various mixtures of liquid extract 40. In the preferred embodiment, the present invention is available in two versions. One version is witch hazel extract entirely comprising the liquid extract 40. Another version includes a combination of witch extract and a proprietary blend of herbs including various healing, soothing, treatment, anti-bacterial, anti-microbial properties, wherein the herbs and witch hazel comprise the liquid extract 40.

### D. Packaging

Each of the pads 20 are preferably stored within a separate packaging 50. The packaging 50 is preferably comprised of clear plastic bags to allow the pads 20 to both flex and to allow an individual to view the pad 20 within the packaging 50. The packaging 50 is also preferably comprised of a fluid impermeable material so as to prevent any of the liquid extract 40 from escaping the pad 20 through the packaging 50. The pads 20 are further preferably vacuum-sealed within the packaging 50 to keep the pads 20 and liquid extract 40 from spoiling and in an original condition. Vacuum-sealing the pads 20 within the packaging 50 may also assist in keeping various healing or soothing liquid extracts 40 from losing any of their healing or soothing properties and also help to keep the liquid extracts 40 moist, fresh and bacteria free.

### E. Container

The container 60 is preferably large enough to hold a plurality of pads 20 separably wrapped in packaging 50. The pads 20 may be also be frozen within the container 60 by placing the container 60 including the pads 20 within a freezer or other cold storage area. The container 60 preferably includes at least one first support 61 and at least one second support 62. The first support 61 and the second support 62 extend upwardly from the lower end of the container 60 at an angle similar to the angle defined between the first outer portion 21 and the center portion 27 and the second outer portion 24 and the center portion 27.

The first outer portion 21 is positioned upon the first support 61 and the second outer portion 24 is positioned upon the second support 62, wherein the supports maintain the first outer portion 21 and the second outer portion 24 in the desired position during storage. It is appreciated that the container 60 may include various other features common to containers 60, such as but not limited to a lid 64.

### F. Operation of Preferred Embodiment

In use, prior to delivering a baby, the user will purchase a container 60 including a plurality of prepackaged and preformed pads 20. The user will place the entire container 60 including the pads 20 within a freezer to allow the pads 20 and liquid extract 40 within the pads 20 to freeze. Shortly after delivering the baby, the user will remove one pad 20 from the container 60 and subsequently from the pad 20's packaging 50.

The pad 20 is then placed around the perineal area of the woman ensuring that the liquid extract 40 is in direct contact with the perineal area and the pad 20 is preferably centered with respect to the woman's body. The pad 20 can either be held in place or the pad 20 may be supported by the underpants of the user, wherein the user puts on their underpants over the pad 20 to secure the pad 20 between the underpants and the perineal area of the user.

The pad 20 will contact the vaginal and perineal areas of the woman and provide healing and soothing. The cold pad 20 helps to reduce swelling, inflammation and pain, and the liquid extract 40 provides comfort and healing. As the pad 20 thaws, the liquid extract 40 is contained in the pad 20 via the first outer portion 21 and the second outer portion 24 absorbing any excess liquid extract 40 and fluid from the perineal area of the woman. When the pad 20 is fully saturated or the woman is through utilizing the pad 20 the pad 20 may simply be disposed of.

What has been described and illustrated herein is a preferred embodiment of the invention along with some of its variations. The terms, descriptions and figures used herein are set forth by way of illustration only and are not meant as limitations. Those skilled in the art will recognize that many variations are possible within the spirit and scope of the invention, which is intended to be defined by the following claims (and their equivalents) in which all terms are meant in their broadest reasonable sense unless otherwise indicated. Any headings utilized within the description are for convenience only and have no legal or limiting effect.

We claim:

1. A postpartum pad, comprising:
a pad including at least one reservoir portion, wherein said at least one reservoir portion extends within an upper surface of said pad;
wherein at least a portion of said pad is comprised of an absorbent material; and
a liquid extract used for healing or soothing of wounds, wherein said liquid extract is positioned within said reservoir portion.

2. The postpartum pad of claim 1, wherein said liquid extract is comprised of witch hazel.

3. The postpartum pad of claim 2, wherein said liquid extract includes a proprietary herbal blend including various healing, soothing, anti-bacterial and anti-microbial properties.

4. The postpartum pad of claim 1, wherein said pad is comprised of a preformed curved shape.

5. The postpartum pad of claim 1, wherein said pad includes a center portion, a first outer portion extending upwardly from said center portion and a second outer portion extending upwardly from said center portion opposite said first outer portion.

6. The postpartum pad of claim 5, wherein said pad includes a first preformed bend between said first outer portion and said center portion and wherein said pad includes a second preformed bend between said second outer portion and said center portion.

7. The postpartum pad of claim 6, wherein said pad is comprised of a preformed U-shaped configuration.

8. The postpartum pad of claim 1, wherein said at least one reservoir portion is concentric with said pad.

9. The postpartum pad of claim 1, wherein said pad is comprised of a 100% organic cotton material.

10. The postpartum pad of claim 1, wherein said pad is comprised of a disposable configuration.

11. The postpartum pad of claim 1, wherein said pad is individually packaged.

12. The postpartum pad of claim 1, wherein said pad is utilized in a frozen state.

13. A postpartum pad, comprising:
a pad including at least one reservoir portion, wherein said at least one reservoir portion extends within an upper surface of said pad;
wherein at least a portion of said pad is comprised of an absorbent material;
wherein said pad is comprised of a preformed U-shaped configuration and wherein said at least one reservoir portion is concentric with said pad; and
a liquid extract used for healing or soothing of wounds, wherein said liquid extract is positioned within said reservoir portion.

14. The postpartum pad of claim 13, wherein said liquid extract is comprised of witch hazel.

15. The postpartum pad of claim 14, wherein said liquid extract includes a proprietary herbal blend including various healing, soothing, anti-bacterial and anti-microbial properties.

16. The postpartum pad of claim 13, wherein said pad is comprised of a disposable configuration.

17. The postpartum pad of claim 13, wherein said pad is comprised of a 100% organic cotton material.

18. A postpartum pad, comprising:
a pad including at least one reservoir portion, wherein said at least one reservoir portion extends within an upper surface of said pad;
wherein said reservoir portion is concentric with said pad;
wherein said pad is comprised of a 100% organic cotton material;
wherein at least a portion of said pad is comprised of an absorbent material;
wherein said pad includes a center portion, a first outer portion extending upwardly from said center portion and a second outer portion extending upwardly from said center portion opposite said first outer portion;
wherein said pad includes a first preformed bend between said first outer portion and said center portion and wherein said pad includes a second preformed bend between said second outer portion and said center portion;
wherein said pad is comprised of a preformed U-shaped configuration and wherein said at least one reservoir portion is concentric with said pad;
wherein said pad is comprised of a disposable configuration;
wherein said pad is individually packaged;
wherein said pad is utilized in a frozen state; and
a liquid extract positioned within said reservoir portion of said pad;
wherein said liquid extract is comprised of witch hazel.

19. The postpartum pad of claim 18, wherein said liquid extract includes a proprietary herbal blend including various healing, soothing, anti-bacterial and anti-microbial properties.

20. The postpartum pad of claim 18, including a container, wherein said container includes a first support and a second support for supporting said first outer portion and said second outer portion in an angled position, wherein said container stores a plurality of said pads.
